(19) United States
(12) Patent Application Publication
(10) Pub. No.: US 2017/0175099 A1
(43) Pub. Date: Jun. 22, 2017

(54) Rapid N-Glycan Release from Glycoproteins using Immobilized Glycosylase Columns

(71) Applicants: Marton Szigeti, San Jose (HU); Akos Szekrenyes, Szentlorinc-Tarcsapuszta (HU); Zsolt Keresztessy, Debrecen (HU); Andras Guttman, San Diego, CA (US)

(72) Inventors: Marton Szigeti, San Jose (HU); Akos Szekrenyes, Szentlorinc-Tarcsapuszta (HU); Zsolt Keresztessy, Debrecen (HU); Andras Guttman, San Diego, CA (US)

(21) Appl. No.: 15/381,039

(22) Filed: Dec. 15, 2016

Related U.S. Application Data
(60) Provisional application No. 62/267,929, filed on Dec. 16, 2015.

(51) Int. Cl. C12N 11/10; C12P 19/04

(52) U.S. Cl. CPC C12N 11/10; C12Y 302/02; C12P 19/04

(57) ABSTRACT

N-glycosylation profiling of glycoprotein biotherapeutics is an essential step in each phase of product development in the biopharmaceutical industry. For example, during clone selection hundreds of clones should be analyzed quickly from limited amounts of samples. On the other hand, identification of disease related glycosylation alterations can serve as early indicators for various pathological conditions in the biomedical field. We describe an improved packed bed column PNGase F functionalized column reactor. The reactor may be packed into a pipette tip column. In some embodiments, a second column or mixed stationary phase may be packed into the column to capture and purify the cleaved glycan prior to analysis. Complete N-glycan removal can be obtained in 10 minutes from all major N-linked glycoprotein types. The approach can be readily applied to automated sample preparation systems, such as liquid handling robots.

### FIELD OF THE INVENTION

The invention relates to an immobilized glycosylase column for the digestion of denatured glycoproteins, purified glycans, cell lysates, and other biological samples. In particular, the invention relates to packed bed columns containing an immobilized glycosylase attached to a substrate.

### BACKGROUND OF THE INVENTION

There is a growing demand in the biopharmaceutical industry for high throughput and large scale N-glycosylation profiling of biotherapeutics in all phases of the product development process. In 2015, monoclonal antibodies represent almost half of the biotherapeutic drugs are with an estimated $70 billion dollar business worldwide, emphasizing the importance of their analysis. On the other hand, glycoproteins play significant roles in cell development, differentiation and interactions. Thus, analysis of the structural changes of the glycan moieties of these highly complex molecules in living systems may give the opportunity to shed light on the courses of diseases leading to new glycosylation based biomarkers however, comprehensive glycoanalytical methods should be able to handle large number of samples in a high throughput fashion demanding rapid and easily to automate glycan analysis tools.

The most frequently used carbohydrate analysis techniques are nuclear magnetic resonance spectroscopy (NMR), mass spectrometry (MS), liquid chromatography (HPLC) and capillary electrophoresis (CE). Capillary electrophoresis features an important advantage as capable to differentiate oligosaccharides based on their molecular shape—even if the mass to charge ratios of two analytes are the same—thus readily separates linkage and positional isomers. In addition, CE requires only nanoliter sample amounts to be injected for full structural analysis, compared to other techniques that need microliter quantities for the same. The workflow of CE based N-glycan analysis includes sample preparation steps of glycan release, fluorophore labeling, purification and pre-concentration prior to separation.

In general, peptide-N4-(N-acetyl-beta-glucosaminyl) asparagine-amidase (PNGase F) is the most frequently used enzyme for N-linked carbohydrate release, due its specific cleavage capability under mild conditions between the innermost core N-acetylglucosamine of the carbohydrate structures and its holding asparagine residue. The reaction is commonly performed in-solution, overnight at 37° C. Efforts have been taken to accelerate this enzymatic reaction by microwave irradiation, ultrahigh pressure or immobilized PNGase F in microfluidics format. Enzyme immobilization has many advantages compared to in-solution based reactions such as long-term operational stability, rapid reaction speed, no enzyme contamination after the reaction and the option of repetitive usage. Pioneering works in the early 1970s successfully utilized immobilized trypsin for rapid protein digestion. Since, enzyme immobilization techniques have been rapidly emerging and used in various fields from research to industrial applications.

In previous work, PNGase F was immobilized onto a methacrylate-based monolithic support prepared in a 250 μm ID fused silica capillary to digest N-glycan removal from glycoproteins followed by off-line MALDI/MS and CE-LIF analysis of released glycans. Both random bound enzyme and tagged enzyme monolith columns were prepared. The monolith PNGase F reactor column was found to digest samples much faster compared to in-solution enzymatic digestion. However, the samples that could be prepared were limited to denatured glycoproteins and purified glycans. In addition, even though sample digestion was rapid, times were still too lengthy. There exists a need for a rapid PNGase F enzyme column that can process a larger variety of samples.

In this invention, PNGase F cloned from Flavobacterium meningosepticum and expressed as a Glutathion-S-Transferase (GST) fusion protein was employed. The GST tagged PNGase F enzyme was immobilized onto glutathione packed bed affinity columns The packed bed column was found to support rapid N-glycosylation digestion up to 6 times faster than the monolith column under identical buffer conditions. In addition, samples such as clarified cell lysates and other biological samples could be reacted. In another embodiment of the invention, a second cleanup column material is mixed or positioned in series downstream from the enzyme column to facilitate capture and purication of the released glycans. The released glycans were labeled via reductive amination using 8-aminopyrene-1,3,6-trisulfonic acid (APTS) and analyzed by capillary gel electrophoresis with laser induced fluorescent detection (CE-LIF).

### SUMMARY OF THE INVENTION

GST tagged PNGase F enzyme and other glycosylases can be immobilized on an affinity columns for rapid and easy-to-automate digestion of N-linked carbohydrates from glycoproteins. Proper immobilization and fast N-glycan release conditions were established. Using PNGase F functionalized columns, almost full deglycosylation can be obtained in 10 minutes compared to in-solution digestion, where twice as much time was required to reach the plateau or compared to a monolith where 6 times as much time was required to reach the plateau. Furthermore, only trace amounts of residual sample remained on the columns after the digestion and washing steps. Excellent reproducibility was obtained during the on-column digestion process between the parallel runs (RSD 2.3%). Interestingly, the on-column digestion showed much faster reaction kinetics than that of its in-solution counterpart. Therefore, the use of these GST tagged PNGase F immobilized columns is a promising approach to fulfill the automation requirement of the high throughput environment of the biopharma industry.

### BRIEF DESCRIPTION OF THE DRAWINGS

FIGS. 1A and 1B depict two different column embodiments of the invention.

FIG. 2 illustrates a glycosylase digestion cycle using a pipette tip column.

FIG. 3 depicts the steps for glycan processing and cleanup using a dual phase column.

FIGS. 4A and 4B illustrate glycosylase digestion and cleanup using two columns operated in series.

FIG. 5 depicts an embodiment of automated sample preparation workflow and plate layout.

FIG. 6 compares the cumulative relative fluorescence units (RFU) values of four major fetuin peaks.

FIGS. 7A, 7B and 7C depict CGE-LIF traces of APTS labeled Immunoglobulin G (a), fetuin (b) and RNase B (c) N-glycans using in solution (A) and on-column (B) digestion. Maltodextrin ladder reference (C).

FIG. 8 depicts a comparison of the residual APTS labeled IgG glycan sample after A) a 15 min column wash, B) the eluted sample and C) the maltodextrin ladder as reference.

### DETAILED DESCRIPTION OF THE INVENTION

In the methods of the invention, a rapid N-deglycosylation method is performed using immobilized enzyme column technology. These enzymes can be used to digest N-linked carbohydrates from glycoproteins. Carbohydrate release from standard glycoproteins can be accomplished using an enzyme immobilized a solid phase within a column. For example, columns packed with exoglycosidases can be used for the digestion and sequencing of released and labeled glycans. These include glycosidases such as PNGase F, PNGase A, silidases, fucosidases, etc. The methods can be performed in a parallel and automated fashion.

The columns used in the methods of the invention can be small packed bed columns made of plastic or metal. The solid phase can be held in place with frits positioned above and below the column bed. The bed size can be in the range of 2 μL, 5 μL, 10 μL, 20 μL, 50 μL or 100 μL. In other embodiments, larger packed bed columns can be used in the range of 200 μL, 500 μL, 1 mL or 2 mL. In certain embodiments, the columns are pipette tip columns. When pipette tip columns are used, the column body can be a variety of sizes including 10 μL, 20 μm, 50 μL, 100 μL, 200 μL, 500 μL, 1 mL or 2 mL. The column bed size can be in the range of 2 μL, 5 μL, 10 μL, 20 μL, 50 μL or 100 μL.

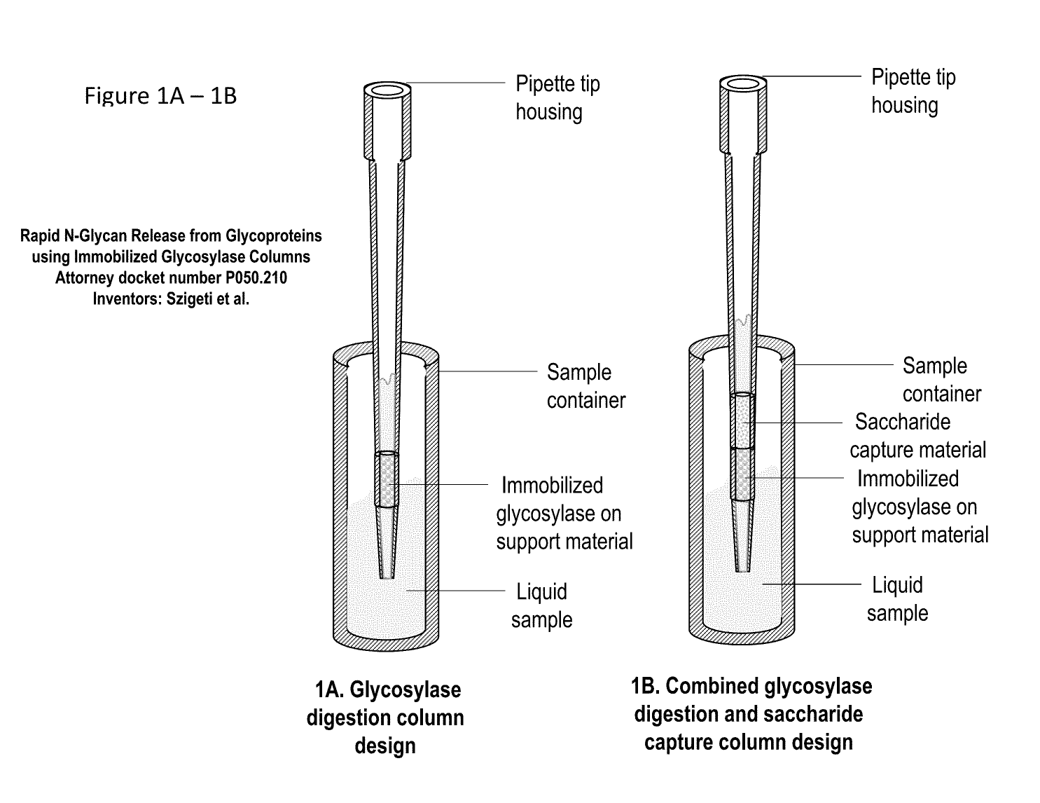

Columns can contain a tagged glycosylation enzyme immobilized on a solid support via their chemical or biological tag. FIGS. 1A and 1B illustrate two different pipette tip column designs. In FIG. 1A, the column contains a single phase bed. In this embodiment, glycosylase enzymes present on the bed digest glycoproteins. In other embodiments, the columns contain two or more different particle types. For example, one column format is comprised of two distinct phases; a glycosylase solid phase and a saccharide capture resin. This configuration is illustrated in FIG. 1B. In another embodiment, two or more particle types can be mixed in the column bed.

A variety of resins can be used within the columns In some embodiments, the resin is a glutathione affinity resin (PhyNexus, San Jose, Calif.). Other resins that can capture tagged molecules include IMAC, FLAG, Streptavidin and others. In addition, any affinity resin that can capture a tagged molecule can be used in the invention.

Enzymes can be immobilized on the column resin via a chemical or biological tag. It can be useful to immobilize glycosylases enzymes on the solid phase. In some embodiments, multiple different glycosylation enzymes are immobilized on the same support material. This allows multiple glycosylation digestion's to be carried out on the same sample within the same column.

Enzymes that can be immobilized are classified as hydrolases, although some types of glycosylase enzymes can also transfer glycosyl residues to oligosaccharides, polysaccharides and other alcoholic acceptors. The glycosylases are subdivided into glycosidases, i.e., enzymes that hydrolyse O- and S-glycosyl compounds (enzyme class 3.2.1) and those that hydrolyse N-glycosyl compounds (enzyme class 3.2.2). Enzymes that hydrolyse a terminal, non-reducing-end glycose (or a well-defined di-, tri- or oligosaccharide) from a glycan, i.e. exoenzymes, are given systematic names based on ‘glycohydrolase’; enzymes that hydrolyse internal glycosidic bonds, i.e. endoenzymes, are given systematic names based on ‘glycanohydrolase’. The same structure is often used when providing accepted names for these enzymes.

In some embodiments the enzymes are tagged. The tag may be attached to the enzyme molecule at any location. The tag location can be chosen in such a way that the immobilized enzyme retains activity. In some embodiments the tag resides on the C-terminal region of the enzyme. The following is a nonlimiting list of glycosidases and glycosyltransferases that can be bound to the solid phase in a packed bed column.
1. Endo F2

Cleaves within the chitobiose (GlcNAc-GlcNAc) core of high mannose and biantennary complex type N-glycans.
2. Endo H

Cleaves within the N,N′-diacetylchitobiose (GlcNAc-GlcNAc) core of high mannose and some hybrid type N-glycans.
3. PNGase A

Cleaves N-linked glycans from glycopeptides, including glycans with α(1,3)-linked core fucose.
4. PNGase F

peptide-N-glycosidase F, peptide-N4-(N-acetyl-β-glucosaminyl)asparagine amidase. Releases intact N-glycans.
5. Sialidases

Releases sialic acids from oligosaccharides.
6. α(1-2)-Mannosidase

Releases α(1-2)-linked mannose residues from the non-reducing terminus of oligosaccharides.
7. α(1-2,3,4,6) Fucosidase

Releases non-reducing terminal α(1-2,3,4,6)-linked fucose from N- and O-glycans.
8. α(1-2,3,6)-Mannosidase

Releases non-reducing terminal α(1-2,3,6)-linked mannose from oligosaccharides.
9. α(1-3,4)-Fucosidase

Releases non-reducing terminal α(1-3,4)-linked fucose from oligosaccharides.
10. α(1-3,4,6)-Galactosidase

The enzyme releases non-reducing terminal α(1-3,4,6)-linked galactose from oligosaccharides.
11. β(1-2,3,4,6) Hexosaminidase

Releases non-reducing terminal β(1-2,3,4,6)-linked N-acetylglucosamine (GlcNAc) and N-acetylgalactosamine (GalNAc) residues from oligosaccharides.
12. β N-Acetylhexosaminidase

Releases all non-reducing terminal β-linked N-acetylglucosamine (GlcNAc) from oligosaccharides. Also known as N-Acetyl-β-D-glucosaminidase, β-N-Acetylglucosaminidase.
13. β N-Acetylhexosaminidase

Releases non-reducing terminal β(1-2,3,4,6)-linked N-acetylglucosamine (GlcNAc) and N-acetylgalactosamine (GalNAc) residues.
14. β(1-3,4)-Galactosidase

Hydrolyzes non-reducing terminal galactose β(1-3) and β(1-4) linkages on oligosaccharides.
15. β(1-4)-Galactosidase

The enzyme releases non-reducing terminal β(1-4)-linked galactose from oligosaccharides and glycoproteins.
16. β(1-4,6)-Galactosidase

Hydrolyses non-reducing terminal Galβ(1-6)GlcNAc and Galβ(1-4)GlcNAc.
17. α(2,3)-Sialyltransferase

For in vitro sialylation of glycoproteins such as monoclonal antibodies.
18. α(2,6)-Sialyltransferase

For in vitro sialylation of glycoproteins such as monoclonal antibodies.
19. β(1,4)-Galactosyltransferase

For in vitro galactosylation of glycoproteins such as monoclonal antibodies.

Column operation can be manual, e.g. with a pipette or automated using a liquid handling system or robot. Columns can be operated using unidirectional or bidirectional flow. When bidirectional flow is used, a pump is attached to the upper end of the column and liquids are aspirated and expelled through the lower end. Liquids can be passed through the column repeatedly or in a single pass.

The columns can be operated at a wide range of temperatures. For example, in some embodiments, the columns are operated at 4° C. while in other embodiments, the columns are operated at higher temperatures, up to 10, 20, 25, 30, 35, 37, 40, 45, 50, 55, 60 and 65° C. and beyond.

For enzyme capture, a solution containing the enzyme is passed through the column. Enzymes can be passed through the column using unidirectional or bidirectional flow. In some embodiments, enzyme immobilization is done in the cold room or at low temperatures. After enzyme immobilization, the columns can be stored in the cold.

Often, the enzyme is tagged prior to capture. A variety of tags can be used including glutathione S-transferase (GST), biotin, HIS, FLAG, Fc and Myc.

The enzyme is usually present in a buffer having a pH in the range of 4 through 9. A nonlimiting list of PNGase F column digestion buffers is shown in table 1.

TABLE 1

| | Useful pH | pKa | pKa | pKa |
|---|---|---|---|---|
| Buffers | Range | (at 20° C.) | (at 25° C.) | (at 37° C.) |
| MES | 5.5-6.7 | 6.16 | 6.1 | 5.97 |
| Bis-Tris | 5.8-7.2 | n/a | 6.5 | 6.36 |
| ADA | 6.0-7.2 | 6.65 | 6.59 | 6.46 |
| ACES | 6.1-7.5 | 6.88 | 6.78 | 6.54 |
| PIPES | 6.1-7.5 | 6.8 | 6.76 | 6.66 |
| MOPSO | 6.2-7.6 | n/a | 6.9 | 6.75 |
| Bis-Tris | 6.3-9.5 | n/a | 6.8, 9.0 | n/a |
| Propane | | | | |
| BES | 6.4-7.8 | 7.17 | 7.09 | 6.9 |
| MOPS | 6.5-7.9 | 7.28 | 7.2 | 7.02 |
| TES | 6.8-8.2 | 7.5 | 7.4 | 7.16 |
| HEPES | 6.8-8.2 | 7.55 | 7.48 | 7.31 |
| DIPSO | 7.0-8.2 | n/a | 7.6 | 7.35 |
| MOBS | 6.9-8.3 | n/a | 7.6 | n/a |
| TAPSO | 7.0-8.2 | n/a | 7.6 | 7.39 |
| Trizma | 7.0-9.0 | 8.2 | 8.06 | 7.72 |
| HEPPSO | 7.1-8.5 | n/a | 7.8 | 6.66 |
| POPSO | 7.2-8.5 | n/a | 7.8 | 7.63 |
| TEA | 7.3-8.3 | n/a | 7.8 | n/a |
| EPPS | 7.3-8.7 | n/a | 8 | n/a |
| Tricine | 7.4-8.8 | 8.16 | 8.05 | 7.8 |
| Gly-Gly | 7.5-8.9 | n/a | 8.2 | n/a |
| Bicine | 7.6-9.0 | 8.35 | 8.26 | 8.04 |
| HEPBS | 7.6-9.0 | n/a | 8.3 | n/a |
| TAPS | 7.7-9.1 | 8.49 | 8.4 | 8.18 |
| AMPD | 7.8-9.7 | n/a | 8.8 | n/a |
| TABS | 8.2-9.6 | n/a | 8.9 | n/a |
| AMPSO | 8.3-9.7 | n/a | 9 | 9.1 |
| CHES | 8.6-10.0 | 9.55 | 9.49 | 9.36 |
| CAPSO | 8.9-10.3 | n/a | 9.6 | 9.43 |
| AMP | 9.0-10.5 | n/a | 9.7 | n/a |

The enzyme can be passed through the column multiple times using unidirectional or bidirectional flow. When bidirectional flow is used, a pause can be incorporated following each aspiration and/or expulsion. Enzyme immobilization is accomplished by repeated unidirectional or bidirectional flow which can continue for some duration. That is, the enzyme solution can be passed through the column repeatedly for up to 1 minute, 2 minutes, 3 minutes, 4 minutes, 6 minutes, 8 minutes, 10 minutes, 15 minutes, 20 minutes, 25 minutes, 30 minutes, 40 minutes, 50 minutes, 60 minutes, 90 minutes, 120 minutes, 240 minutes or up to 250 minutes. In some embodiments, enzyme capture can be performed in the cold, e.g. 4° C. Capture may be performed at higher temperature provided the enzyme is stable. Temperatures of up to 10, 20, 25, 30, and 35° C. may be used.

Columns comprised of immobilized enzymes can be used for enzymatic digestion of glycoprotein samples. The samples can contain a single glycoprotein or mixtures thereof. Often, the glycoproteins are denatured prior to enzymatic digestion. The glycoprotein may be denatured with heat, salts, chemicals or solvents. The amount of glycoprotein digested can be in the range of 5μg, 10 μg, 20 μg, 50 μg, 100 μg, 200 μg, 400 μg, 600 μg, 800 μg or 1000 μg. The concentration can be in the range of 0.01, 0.1, 0.5, 1, 2, 3, 4, or 5 mg/mL.

For the digestion step, the denatured glycoproteins can be aspirated and expelled through the lower end of the column In some embodiments, the flow for digestion may be unidirectional. The enzymatic digestion step can be performed at a variety of temperatures. That is, the enzymatic digestion step can be performed in the range of 20° C. to 60° C., 30° C.-55° C. or 40° C.-50° C. In certain embodiments, digestion is performed at 50° C.

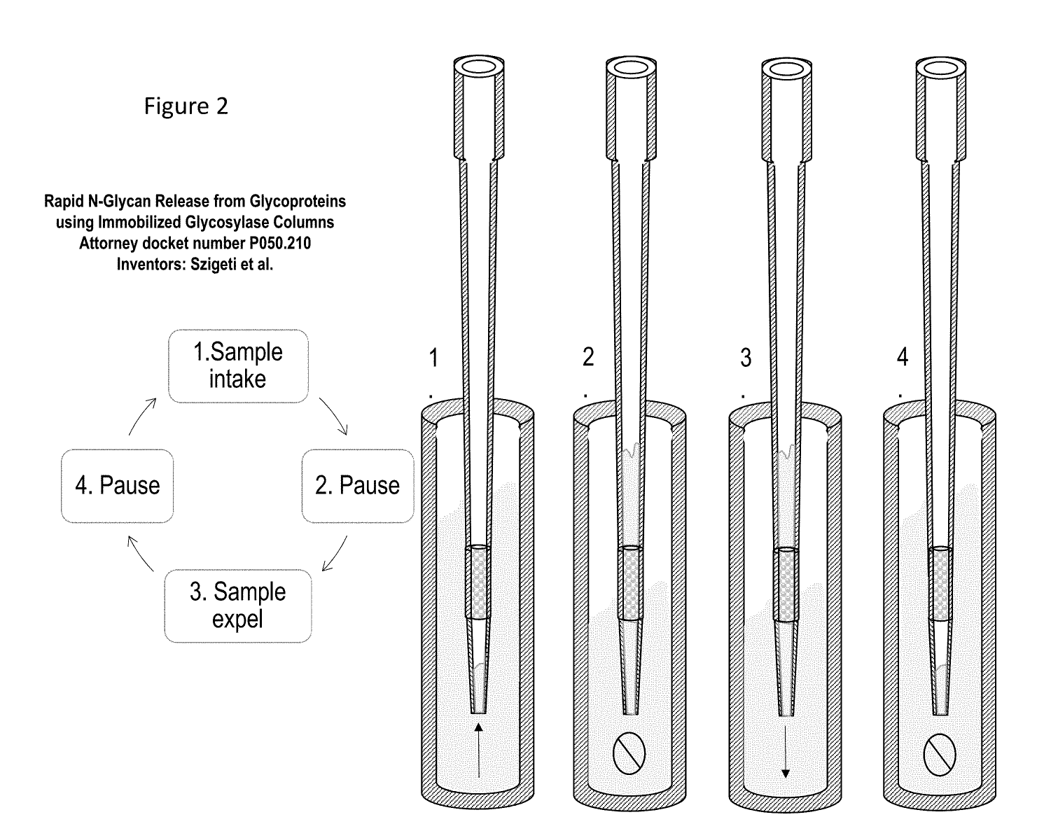

A basic digestion cycle is shown in FIG. 2. First, the column is placed in a liquid sample. The sample is aspirated in step 1. Liquid passes through the resin bed and fills the column When the column is filled, the sample can be kept in the column for a short period. In these embodiments, a pause can be used following aspiration and proceeding expulsion. Similarly, a pause can be incorporated following expulsion. Pauses can be 1 second, 2 seconds, 3 seconds, 4 seconds, 5 seconds, 10 seconds, 15 seconds, 20 seconds, 25 seconds or longer. After the parts, the sample passes through the resin bed again while it is expelled from the column. This aspiration/expulsion cycle can be repeated multiple times and tell complete digestion is achieved.

The flow rate used for the enzymatic digestion step can vary. The flow rate can be 0.05 mL/min, 0.1 mL/min, 0.2 mL/min, 0.4 mL/min, 0.8 mL/min, 1 mL/min, 1.2 mL/min, 2 mL/min, 4 mL/min, 5 mL/min, 6 mL/min, 8 mL/min or 10 mL/min.

Depending on the conditions, the digestion reaction can be allowed to proceed for varying lengths of time. On-column digestion can proceed for 15 seconds, 30 seconds, 1 minute, 2 minutes, 3 minutes, 4 minutes, 5 minutes, 7 minutes, 10 minutes, 12 minutes, 14 minutes, 20 minutes, 30 minutes, or up to 40 minutes.

The digestion process can be very efficient; very little sample remains undigested. For example, the percentage of undigested sample can be less than 1%, less than 2%, less than 3%, less than 4%, less than 5%, less than 6%, less than 8%, less than 10%, less than 15% or less than 20%.

TABLE 2

| | Example | Range |
|---|---|---|
| Device | | |
| Pipette tip volume | 1 mL | 20, 50, 200, 1000, |
| | | 2000 μL |
| Active volume | 10 μL bed size | 2, 5, 10, 20, 50, 100 μL |
| Required tools for | Automated pipette | Manual pipette, liquid |
| digestion | (liquid handling system) | handling robotic |
| | Heat block | Room temp, heated block |
| Immobilize Enzyme | | |
| Buffer | 20 mM Tris-HCl | any buffer pH 4, 5, |
| | (pH 7.30) | 6, 7, 8, 9 |
| Immobilization | 3 h (4° C.) 60 μL/min | 1, 2, 3, 4, 6, 8, 10, 15, |
| time | (intake-15 sec | 20, 25, 30, 40, 50, 60, |
| | pause-expel base cycles) | 90, 120, 240, 250 min |
| Digestion | | |
| Protein amount | 100 μg (1 mg/ml) | 5, 10, 20, 50, 100, 200, |
| | | 400, 600, 800, 1000 μg |
| Digestion | 50° C. | 20, 25, 30, 35, 37, 40, |
| temperature | | 45, 50, 60° C. |
| Flow rate | 5 mL/min (intake-15 sec | 0.05, 0.1, 0.2, 0.4, |
| | pause-expel cycles) | 0.8, 1.2, 2, 4, 5, |
| | | 6, 8, 10 mL/min |
| Digestion time | 10 min | 1, 2, 3, 4, 5, 7, 10, 12, |
| | | 14, 20, 30, 40, min |
| Sample loss | <1% | <1 2, 3, 4, 5, 6, 8, |
| | | 10, 15, 20%, |
| Digestion buffer | 20 mM Tris-HCl | any buffer pH 4, 5, 6, |
| | (pH 7.30) | 7, 8, 9 |
| Specification | | |
| De-glycosylation | 10 min | 1, 2, 3, 4, 5, 7, 10, 12, |
| time & capacity | | 14, 20, 30, 40, min |
| (100 μg RNase B) | | |
| Recovery | >99% | >50, 60, 70, 80, 90, 95, |
| | | 96, 97, 98, 99% |
| Automation option | YES | Manual or automated |
| High-throughout | YES | Single wells, vials |
| capability and | | or plates |
| well plate format | | |
| Combined glycan | YES | Serial or dual column |
| clean up option | | operation |
| Sample | Denatured glycoprotein | Native or denatured |
| | | glycoproteins; purified |
| | | glycans; cell lysates, |
| | | complex biological sam- |
| | | ples; clarified harvest etc. |

After glycoprotein cleavage, capture and purification of the cleaved glycan moieties can be accomplished using a glycan capture resin. This glycan capture resin can be housed in a second column such that glycan capture and purification is performed after the digestion step and prior to detection. Alternatively, the glycan capture resin can reside in the same column as the immobilized glycosylated as described above and shown in FIG. 1B. Capture and purification of the glycan is performed with the same column with the dual phases. However, the enzymatic phase is active first and then in a second step the capture and purification phase is employed.

This format allows the digestion/release of oligosaccharides and the capture of the processed oligosaccharides from the same sample. For example, using a sample that contains glycoproteins the N-glycans can be released from the glycoproteins and then the released N-glycan can be captured with the same column. The sample can then be cleaned up and eluted to another solution for further processing. This invention allows much faster oligosaccharide processing (release or sequencing) of the target oligosaccharides with the built in capture and clean up reducing significantly the sample preparation time.

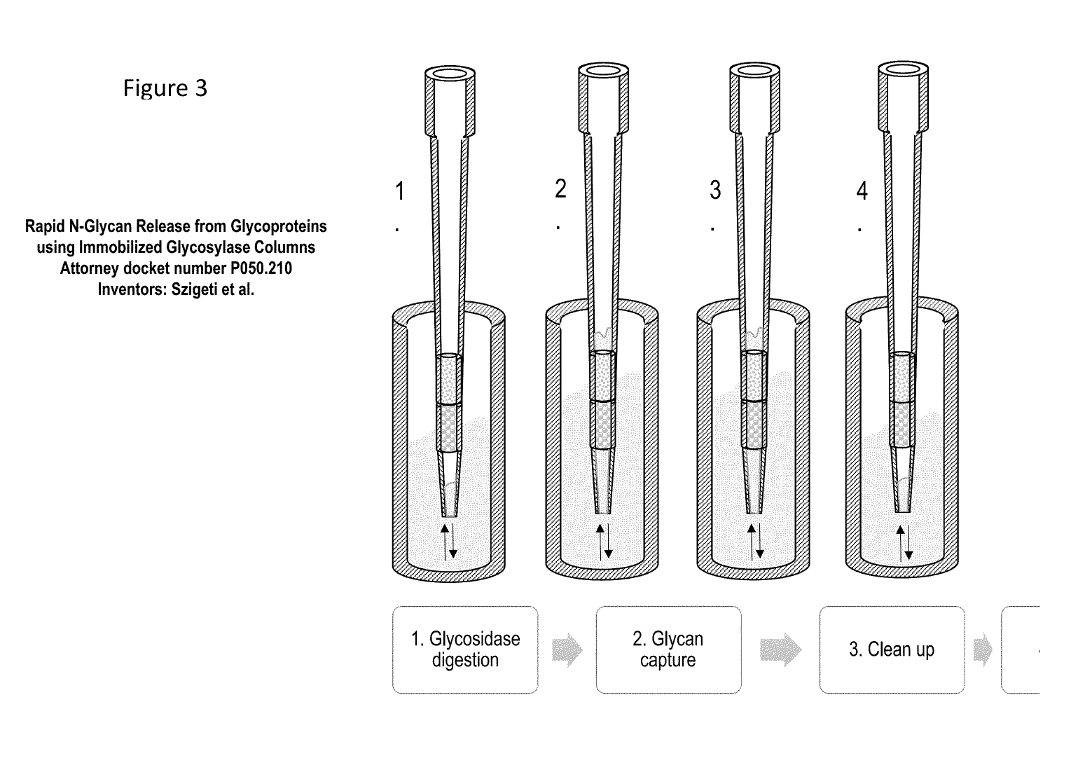

FIG. 3 shows the major steps of glycan processing using a combined or dual phase column. In step 1, the column is placed in a liquid sample and the sample is circulated up and down to cleave the glycan moiety from the glycoprotein. After digestion is complete, additional reagents are added to the sample to allow capture of the glycan on the glycan capture resin. Again, repeated aspiration and expulsion steps are used. Next, a wash solution is circulated through the resin bed's repeatedly to remove any nonspecifically bound molecules. This wash step may be repeated several times with fresh or different wash solutions. Finally, the column is immersed in an elution solution to remove the glycans from the capture support material.

The dual phase column may be constructed in two ways. In some embodiments, the two phases are positioned in the same pipette tip structure with the column positioned in series. In some embodiments, the two phases are mixed and positioned into a single packed bed.

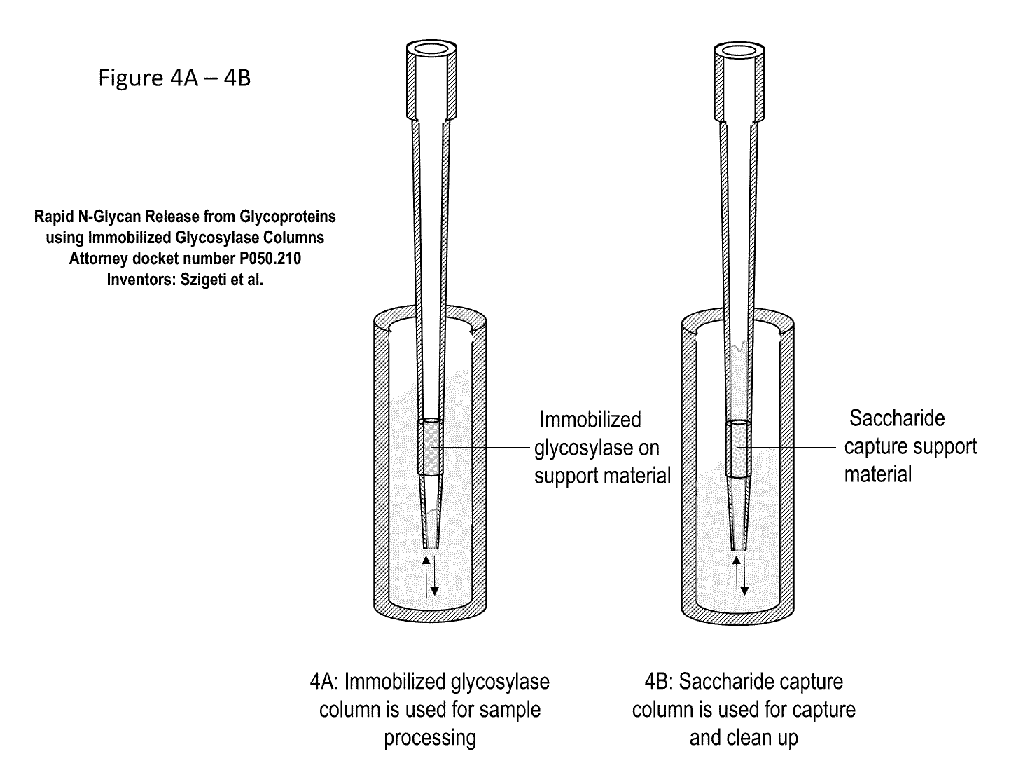

FIG. 4A and 4B illustrate glycosylase digestion and clean up with two columns operated in series. The sample processing and cleanup procedures are the same as those described above. In FIG. 4A, the sample is first processed with the immobilized glycosylase column and then the column is removed from the sample solution. Next, a capture solvent such as acetonitrile, ethanol, isopropyl alcohol or you THF is added to the sample and it is processed in a saccharide capture column shown in FIG. 4B. A capture solvent is defined herein as the solvent that allows saccharide capture. A wash solution and eluent can be used with the saccharide capture column to purify and elute the glycan moieties.

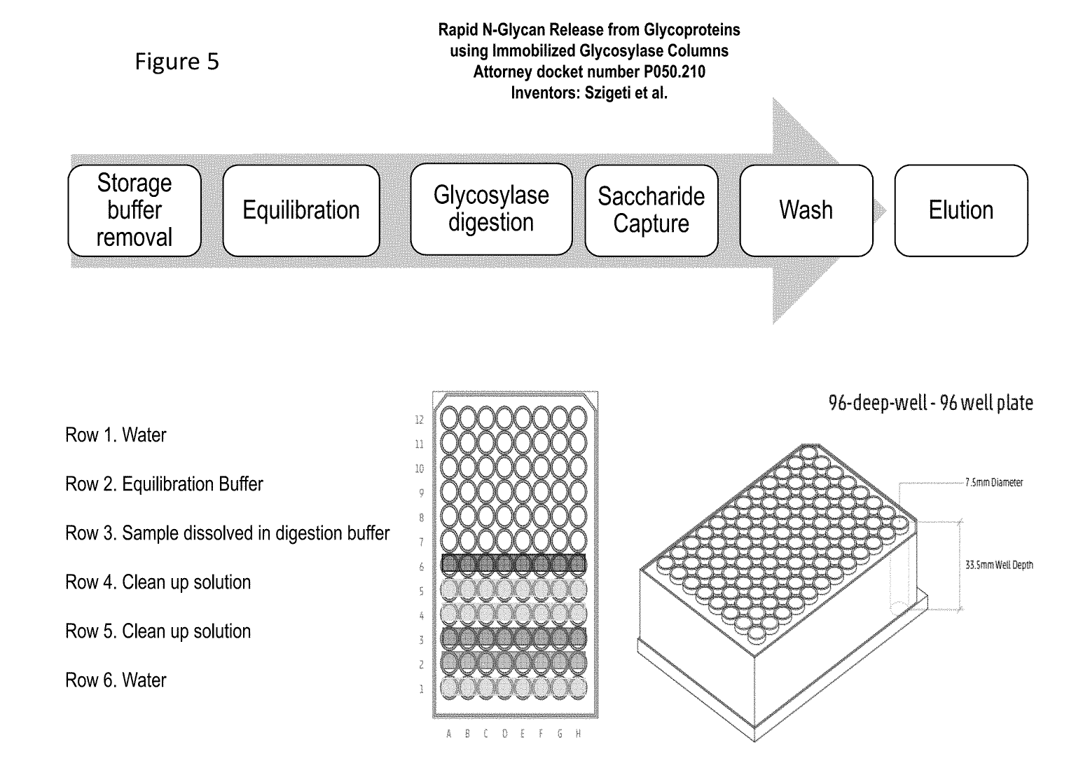

In some embodiments, multiple samples can be processed in parallel. In these embodiments, processing can be performed in an automated fashion. The pipette tip column format allows complete automation of the complex sample preparation process using liquid handling robots and 96 or 386 well plate format. FIG. 5 depicts the general plate and layout for automated sample preparation in a 96-well format. The first row the plate is filled with conditioning solution that is applied to remove storage buffer and components from the column.

The second row is filled with an equilibration buffer used to equilibrate the column before sample processing. The next row contains the sample dissolved in the appropriate sample buffer.

The optimal pH for enzyme activity is 8.6. However, the activity is stable for a wide variety of conditions and reagents. PNGase F maintains 60% activity from pH 6.0 to pH 9.5. It is able to de-glycosylate in the absence of denaturants, but needs extensive incubation and larger amounts of the enzyme to cleave native proteins.

An example of methods of the invention follows. The PNGase F enzyme only works when the target proteins are denatured. First, the sample is denatured using the appropriate solutions at 65° C. for 10 minutes. Then, the sample is diluted with a buffer to a volume of 100 μL. For columns having a bed volume between 10 and 80 μL, 100 μL is a good volume for the sample. The sample is diluted with the buffer that was used for the enzyme immobilization. Next, a heating block is placed under the automated pipettor and set to 50° C., and the lower end of the pipette tip column is immersed in the sample. Aspiration and dispense speed was set to 5 mL/min, and the reason for the pauses in between was to ensure the time for the sample to go through the whole bed up and down. This cycle went for 10 minutes. The PNGase F cleaved the glycans from the proteins.

The next steps relate to CE analysis, because most glycans do not have charges or fluorophore or autofluorescence properties for detection. The magnetic bead CleanSEQ is used to capture the glycans and to get rid of any contamination and the protein residue. It captures the sugars in organic phase (MeCN), and releases them in the aqueous phase. The glycans were captured after the digestion step, and labelled using APTS dye with the necessary solutions on the beads. That gives the glycans charges so that they can be pulled in the capillary using an electric field and the fluorophore group for the detection. Finally, a PhyNexus Normal Phase tip is used to remove the excess dye from the sample. It is necessary, because the excitation of the sample-APTS conjugate is close to the APTS excitation itself. Therefore it gives big, tailing signal upfront and could corrupt the measurements.

In summary, it was discovered that the packed bed pipette column could digest all N-glycan subclasses of neutral, sialylated and high mannose at the rate of 10 μg/min or higher which is at least six times faster than the monolith capillary digesting the same glycan with the same enzyme under the same buffer conditions at the rate of 1.6 μg/min. In addition, the monolith only digested the high mannose N-glycans at this rate; the other and more important N-glycan digestion was not shown. It was further discovered that the kinetic rate of the enzyme-immobilized packed bed column could be increased to 100 μg/min or even 200 μg/min by increasing the bed volume or increasing the flow rate. The kinetic rate can be greater than 2μg per minute, 10 μg per minute, 50 μg per minute, 100 μg per minute or 200 μg per minute.

### EXAMPLES

### Example 1.

### Immobilization of GST tagged PNGase F

For the immobilization process, 1 mL size glutathione affinity resin containing columns were used with 10 μL bed volume (PhyNexus, San Jose, Calif., USA). Pipetting was done automatically using 1000+ pipette heads with the controller software (Capture-Purify-Enrich, Version 2.2.3, PhyNexus). First, the storage solution was removed by applying 0.06 mL·min⁻¹ flow rate. Then, the washed with 1 mL of 20 mM Tris-HCl buffer (pH 7.3) at room temperature using 1 mL full aspiration and dispense at 0.06 mL·min⁻¹ flow rate once, and three times at 0.50 mL·min⁻¹. After washing the columns, 1.0 μL of GST PNGase F (16 IUB mU/mL) enzyme was mixed with 1 mL of 20 mM Tris-HCl buffer (pH 7.3) and aspirated/dispensed for 3 hours using 0.06 mL·min⁻¹ flow rate at 4° C. for compete binding. The columns were stored at 4° C. filled with fresh 20 mM Tris-HCl buffer (pH 7.3).

### Example 2.

### Glycan Release and Derivatization

Sample preparation was accomplished by using 10 μL of 10 mg/mL standard glycoprotein solution (IgG, fetuin, RNase B) in HPLC grade water, denatured by the addition of 1 μL of 5% SDS and 1 μL of 50 mM of DTT at 65° C. for 10 minutes. After the denaturation step, 2.5 μL of 10% NP-40 and 85.5 μL of 20 mM Tris-HCl buffer (pH=7.3) were added resulting 100 μL of total sample volume. The enzymatic digestion was performed using 10 μL bed volume 1 mL size GST-PNGase F immobilized glutathione columns at 50° C. with 600 μL aspiration/dispense volume at 5 mL·min⁻¹ flow rate with 15 sec pauses between steps. After the enzymatic digestion, 900 μL of acetonitrile (100%) was added to the sample resulting 90.0% final acetonitrile concentration, and added to 200 μL of Agencourt CleanSEQ beads (storage solution removed) and vortexed at 2,500 rpm for 10 sec for carbohydrate partitioning. After 1 minute wait, the beads were pulled to the side of the vials by a magnet, and the supernatant was removed. The samples were then labeled on the beads by adding 9 μL of APTS (40 mM in 20% acetic acid) and 1 μL of NaBH₃CN (1 M in THF) followed by incubation at 50° C. for 1 hour. After the labeling reaction, the reaction was stopped by the addition of 100 μL of water and the sample was vortexed at 2,500 rpm for 10 sec. Then, the beads were pulled to the side of the vials by a magnet, and a supernatant was transferred to a new vial. Next, 900 μL of acetonitrile was added and the samples were aspirated/dispensed for 25 times at 5 mL·min⁻¹ speed using PhyNexus Normal Phase columns. The labeled glycans were then eluted by 20 μL of water and analyzed by CGE-LIF.

### Example 3.

### Capillary Gel Electrophoresis

Capillary gel electrophoresis with laser induced fluorescence detection (CGE-LIF) was performed on a PA800 Plus Pharmaceutical Analysis System (SCIEX). For rapid sample analysis, 20 cm effective length NCHO capillary (30 cm total length, 50 μm ID) (SCIEX) was filled with the separation gel buffer of 1% PEO (MW 900 kDa) in 25 mM lithium acetate buffer (pH 4.75). The separation voltage was 15 kV in reversed polarity mode (cathode at the injection side, anode at the detection side) resulting in 500 V/cm applied electric field strength. The samples were pressure injected by 1 psi (6.89 kPa) for 5 seconds. The 32 Karat (version 9.1) software package (SCIEX) was used for data acquisition and analysis.

### Example 4.

### On-Column Digestion Versus in Solution Digestion

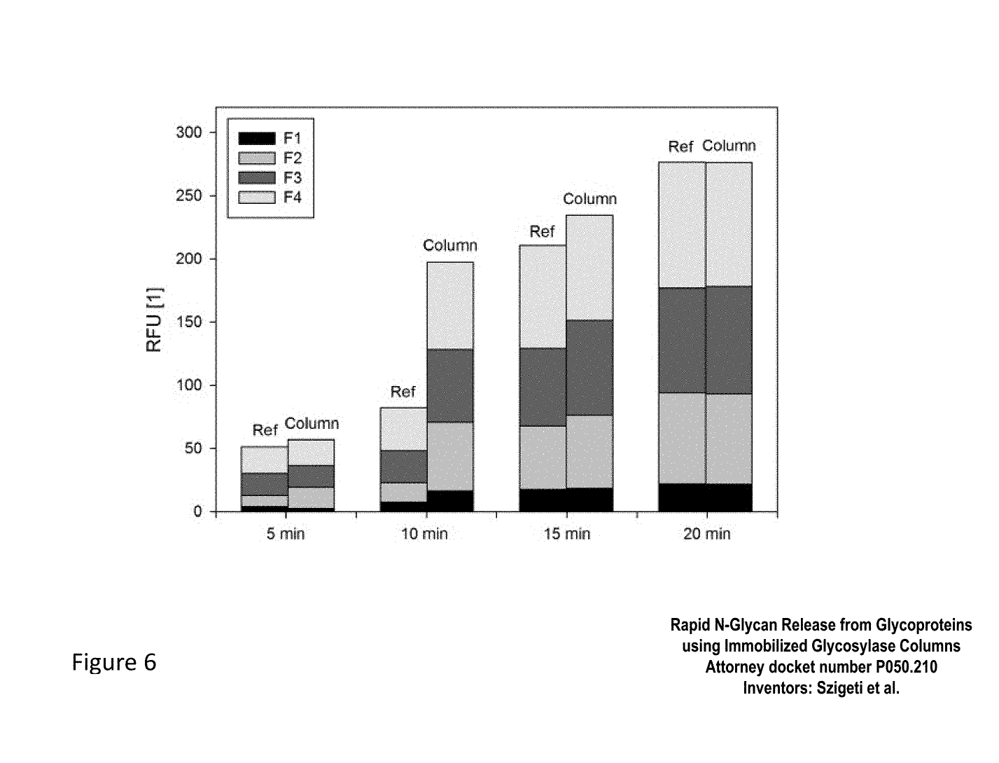

The effect of digestion time was evaluated both for on-column and in-solution reaction between 5 and 20 min in 5 min intervals. Fetuin was used as standard glycoprotein in these experiments. The cumulative RFU values of the four major fetuin glycan peaks (F1-F4) are shown in FIG. 6 as the function of the digestion time for both reaction conditions.

The results show completely different release characteristics for the two digestion techniques. While using conventional in-solution digestion the amount of digested glycans increase in a concave fashion and reach the plateau at 20 min, the on-column digestion method shows a convex curve with very rapid increase reaching approximately 70% of the maximum N-glycan release in 10 min reaction time. Please note that peak distribution (black and gray zones representing F1-F4 peaks) remained the same during the time course.

### Example 5.

### A Valuation of Three Major and Glycosylated Some Subclasses

Glycoprotein samples representing the three major N-glycosylation subclasses:

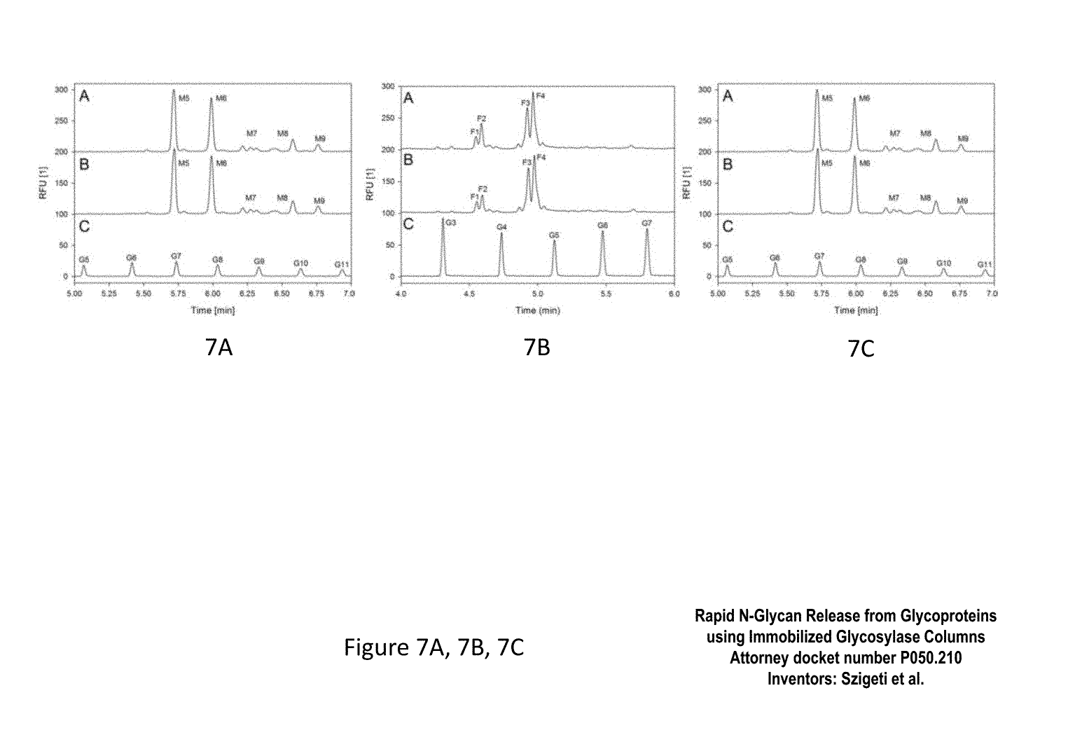

immunoglobulin G, which contains mostly complex neutral biantennary and a few sialylated glycans; fetuin, with mostly highly sialylated structures; and RNase B possessing high mannose type oligosaccharides were evaluated to understand possible structural release bias. The released and APTS labeled glycans from the three types of glycoprotein samples were analyzed by CGE-LIF. FIGS. 7A through 7C compare the data obtained by in-solution glycan removal (A traces) and on-column digestions (B traces) using identical sample preparation parameters in 20 min digestion times showing practically identical traces between on-column and in-solution digestions for the three glycoprotein sample types examined However, as FIG. 6 shows that with the use of PNGase F functionalized packed bed columns, 70% N-deglycosylation was obtained in approximately 10 minutes and negligible differences were observed in peak areas, peak area percentages and migration time values (see numerical comparison in Supplementary Tables 1-3).

### Example 6.

### Recovery and Reproducibility

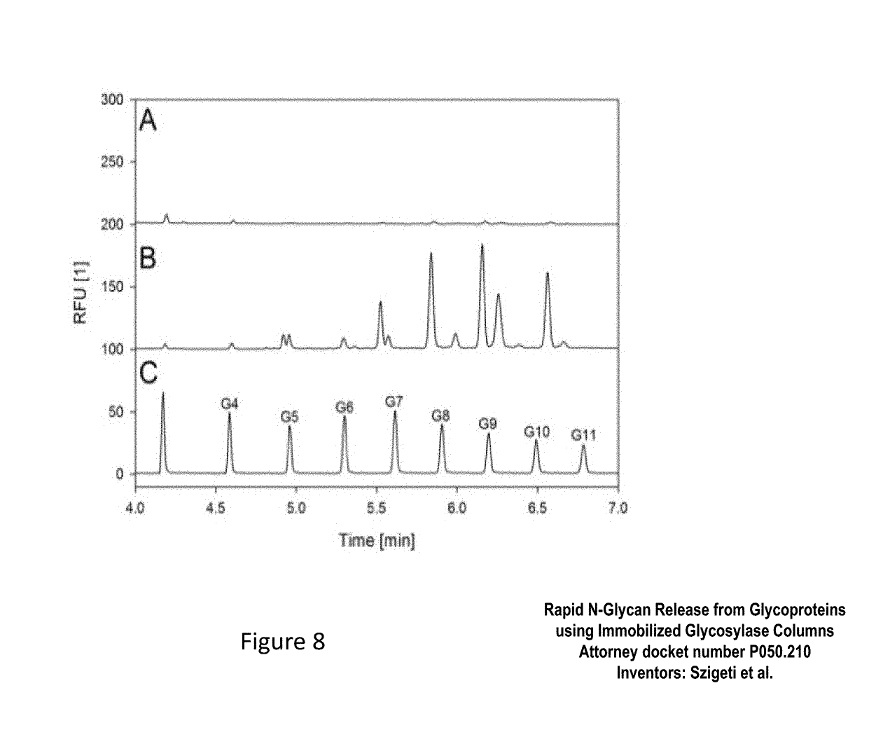

The amount of any possibly remaining residual sample on the columns after the digestion process was also examined The small (10 μL) resin-bed structure enabled relatively high speed aspiration/dispense cycles, while maintaining the high efficiency of the digestion performance. The five times higher flow rate compared to 80 μL resin-be column ensured intense contact between the sample and the immobilized enzyme resulting in apparently no sample leftover on the columns. Trace A in FIG. 8 show only very minimal residual sample after repeated water washing steps (30 cycles in 15 min), compared to the eluted IgG N-glycan sample shown by trace B. The average peak area of the residual sample was less than 1% of the total eluted peak area.

The reproducibility of the digestion process with the immobilized enzyme columns was also assessed. Sample preparation and digestion of IgG was performed using three GST-PNGase F functionalized columns The results of the CGE-LIF measurements were evaluated in respect to their peak area, peak area percentage and migration times and compared among the three consecutive runs. Table 1 depicts the data of the 10 major IgG glycan peaks (see FIG. 7A) revealing no significant difference between the areas, area percentages and migration times among the three parallel runs.

FIG. 8. Comparison of the residual APTS labeled IgG glycan sample after 15 min column wash (A) and the eluted sample (B). Maltodextrin ladder reference (C). Separation conditions were the same as in FIG. 2.

TABLE S1
Comparison of peak areas and migration times using in solution
digestion (A) and column digestion (B) using Immunoglobulin G

| | | Reference - A | | | Column digestion - B | | | | |
|---|---|---|---|---|---|---|---|---|---|
| | | | | Mig. | | | Mig. | Difference (%) | |
| | | | Area | time | | Area | time | | Mig. |
| No. | Peak | Area | % | (min) | Area | % | (min) | Area | Time |
| 1 | FA2G2S2 | 15.80 | 2.42 | 4.92 | 15.63 | 2.34 | 4.92 | 1.077 | 0.089 |
| 2 | FA2BG2S2 | 15.00 | 2.29 | 4.95 | 14.56 | 2.18 | 4.95 | 3.047 | 0.006 |
| 3 | FA2[3]G1S1 | 13.88 | 2.12 | 5.29 | 13.57 | 2.03 | 5.29 | 2.289 | 0.068 |
| 4 | FA2G2S1 | 58.71 | 8.98 | 5.52 | 56.53 | 8.45 | 5.52 | 3.869 | 0.075 |
| 5 | FA2BG2S1 | 16.64 | 2.55 | 5.57 | 16.31 | 2.44 | 5.57 | 2.028 | 0.075 |
| 6 | FA2 | 139.17 | 21.28 | 5.84 | 136.88 | 20.47 | 5.83 | 1.673 | 0.073 |
| 7 | FA2B | 23.46 | 3.59 | 5.99 | 23.23 | 3.47 | 5.98 | 1.019 | 0.066 |
| 8 | FA2[6]G1 | 151.01 | 23.09 | 6.16 | 152.60 | 22.82 | 6.15 | 1.045 | 0.075 |
| 9 | FA2[3]G1 | 103.59 | 15.84 | 6.26 | 103.77 | 15.52 | 6.25 | 0.174 | 0.076 |
| 10 | FA2G2 | 116.62 | 17.83 | 6.56 | 118.64 | 17.74 | 6.56 | 1.705 | 0.066 |

TABLE S2
Comparison of peak areas and migration times using in solution
digestion (A) and column digestion (B) using Fetuin sample

| | A - Reference | | | B - Column digestion | | | Difference (%) | |
|---|---|---|---|---|---|---|---|---|
| | | | Mig. | | | Mig. | | Mig. |
| | | Area | time | | Area | time | | time |
| Peak | Area | % | (min) | Area | % | (min) | Area | (min) |
| F1 | 29.32 | 7.86 | 4.55 | 27.17 | 7.28 | 4.55 | 7.916 | 0.105 |
| F2 | 47.89 | 12.83 | 4.59 | 42.86 | 11.48 | 4.59 | 11.733 | 0.101 |
| F3 | 110.13 | 29.51 | 4.92 | 115.19 | 30.87 | 4.93 | 4.397 | 0.174 |
| F4 | 185.84 | 49.80 | 4.96 | 190.17 | 50.96 | 4.97 | 2.274 | 0.184 |

TABLE S3
Comparison of peak areas and migration times using in solution
digestion (A) and column digestion (B) using RNase B sample

| | A - Reference | | | B - Column digestion | | | Difference (%) | |
|---|---|---|---|---|---|---|---|---|
| | | | Mig. | | | Mig. | | Mig. |
| | | Area | time | | Area | time | | time |
| Peak | Area | % | (min) | Area | % | (min) | Area | (min) |
| M5 | 184.36 | 38.40 | 5.72 | 187.67 | 38.12 | 5.72 | 1.77 | 0.046 |
| M6 | 161.29 | 33.60 | 5.99 | 167.53 | 34.03 | 5.99 | 3.73 | 0.022 |
| M7 | 50.28 | 10.47 | — | 49.75 | 10.11 | — | 1.06 | — |
| M8 | 58.00 | 12.08 | — | 60.33 | 12.25 | — | 3.87 | — |
| M9 | 26.13 | 5.44 | 6.76 | 27.03 | 5.49 | 6.76 | 3.36 | 0.012 |

1. A method for glycan purification comprising:
a) providing a pipette tip column comprised of an open lower end and a packed resin bed, wherein the packed resin bed is comprised of an immobilized glycosylase;
b) providing a sample comprised of glycoproteins;
(c) aspirating the glycoprotein sample through open lower end of the pipette tip column into the packed resin bed;
(d) expelling the glycoprotein sample through the open lower end of the pipette tip column; and
(e) repeating steps (c) and (d), whereby the immobilized glycosylase cleaves the glycan moieties from the glycoprotein in the sample.

2. The method of claim 1, wherein step (e) is performed for 10 minutes or less.

3. A method for glycan purification comprising:
a) providing a pipette tip column comprised of an open lower end and a dual phase packed resin bed, wherein the dual phase packed resin bed is comprised of an immobilized glycosylase and a saccharide capture resin;
b) providing a sample comprised of glycoproteins;
(c) aspirating the sample through open lower end of the pipette tip column into the dual phase packed resin bed;
d) expelling the sample through the open lower end of the pipette tip column;
(e) repeating steps (c) and (d), whereby the immobilized glycosylase cleaves the glycan moieties from the glycoprotein in the sample;
(f) adding a capture solvent to the sample;
(g) aspirating the sample through open lower end of the pipette tip column into the dual phase packed resin bed; and
(h) expelling the sample through the open lower end of the pipette tip column;

4. The method of claim 3, wherein step (e) is performed for 10 minutes or less.
